(19) United States
(12) Patent Application Publication
(10) Pub. No.: US 2005/0171401 A1
(43) Pub. Date: Aug. 4, 2005

(54) Endoscope tip pressure feedback control system for insufflated fluids and gases

(76) Inventor: John Anthony Woltjen, Mission Hills, KS (US)

Correspondence Address: Dr. John A. Woltjen, 2601 West 64th Street, Mission Hills, KS, 66208, US

(21) Appl. No.: 10/771,030

(22) Filed: Feb. 3, 2004

(51) Int. Cl. A61B 1/00

(52) U.S. Cl. 600/118; 600/117

(57) ABSTRACT

Endoscopes are designed to deliver gas and fluid to body cavities or hollow organs during examinations and surgery to accomplish desired tasks. The delivery of gas and fluid potentially increases the body cavity or hollow organ ambient pressure to an injurious level. This invention senses the body cavity or intra luminal organ pressure and stops the flow of gas and/or fluid before injury can occur. Gas and fluid flow is re-established when the ambient pressure falls to a safe level.

### CROSS REFERENCES

Not applicable.

### STATEMENT OF FEDERALLY SPONSORED RESEARCH

This invention was not a product of federally sponsored research or development.

### REFERENCE TO SEQUENCE LISTING

Not applicable.

### BACKGROUND OF THE INVENTION

1. Field of Invention

This invention has to do with endoscopes used in the examination and surgery of body cavities and hollow organs. These instruments are capable of delivering gases and fluids into the body cavity or hollow organ during their use. Heretofore, there was not an appreciation that the pressure used to deliver the gases or fluids may cause pressure injury (barotrauma) to the body.

2. Description of Prior Art

Water and air are delivered from a light source through the endoscope into the body cavity or hollow organs by an integrated design. Typically, an air pump is located in the light source. It pressurizes a water bottle with air above the water line. A trumpet-style value on the endoscope is the controller and it has three states. In the inactive state no water or air is delivered to the body cavity or hollow organ. Air escapes through a low resistance pathway to the atmosphere via a hole in the trumpet-style valve surface. When the air hole is covered by the operator's finger, air flows from the pump to the top of the water bottle and through the endoscope into the body cavity or hollow organ. When the trumpet-styled valve is depressed by the operator's finger, air pressurizing the top of the water bottle forces water from the bottom of the water bottle through the endoscope into the body cavity or hollow organ.

There are alternative ways to control the delivery of gases and fluids to the body cavity via the endoscope. The air water delivery system could be controlled by electromagnetic valves instead of mechanical valves. Compressed gas or air in a storage tank controlled by mechanical or electromagnetic valves may be delivered into the body cavity or hollow organ through the endoscope.

None of these systems have feedback control mechanisms to protect the body cavity or hollow organ from barotrauma. The pressure needed to overcome the internal resistance to gas and fluid flow through the endoscope is high enough to potentially injure the body cavity or hollow organ.

3. Barotrauma of the Colon

Recently, a manuscript “A Retrospective Study of Cecal Barotrauma Analyzing Colonoscopic Air Flow and Pressure” was submitted for publication to Gastrointestinal Endoscopy. In this research study there were a total 3000 colonoscopies divided into two groups of 1500. In one group there were no barotrauma induced colon perforations and in the other there were 4, p<0.022. The colonoscopes used in the group with colon perforations delivered significantly higher pressures and air flows to the colon.

The conclusion of the research was there are some patients because of altered anatomy who cannot eliminate insufflated air at a minimum rate of 1.46 liters per minute at 80 mmHg pressure and they are at risk for colon rupture. This led to the idea that an endoscope tip pressure feedback control system would protect these patients from injury.

### BRIEF SUMMARY OF THE INVENTION

This invention monitors the pressure inside the body cavity. When a preset pressure is exceeded further delivery of gas (and if desired, fluid) will stop until the pressure falls below the predetermined value. During the period of no flow the operator can choose to hear an audible alarm.

### BRIEF DESCRIPTION OF FIGURES

FIG. 1: The typical tip of the endoscope showing location for pressure transducer.

FIG. 2: Block diagram of pressure feedback control of gas or fluid flow delivered to the body cavity by the endoscope.

### DETAILED DESCRIPTION OF THE INVENTION

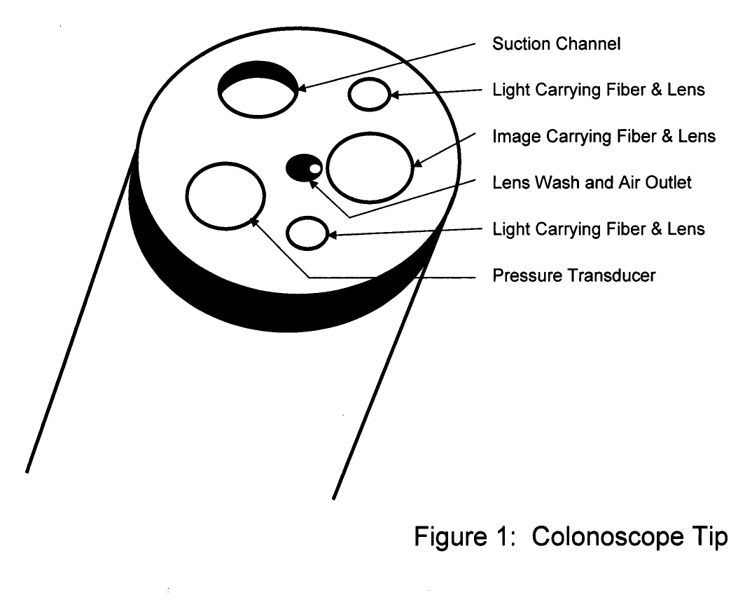

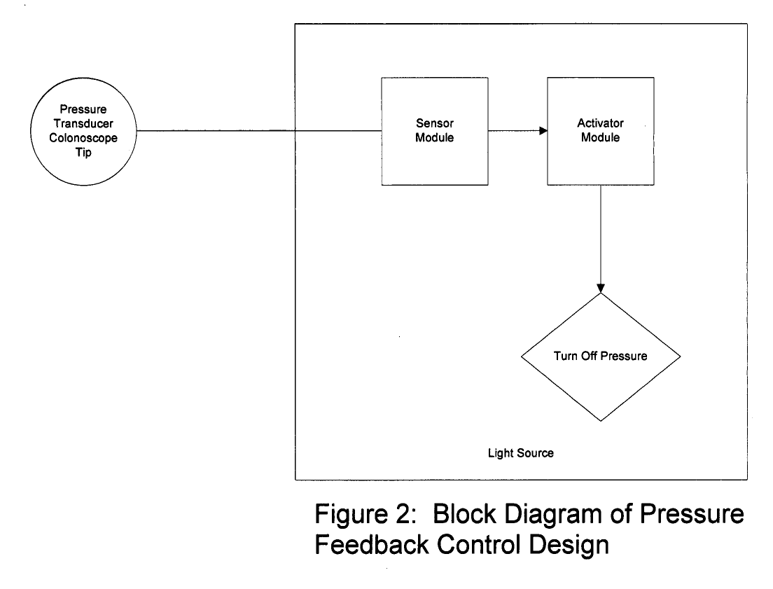

This invention uses a pressure transducer to sense ambient intra organ pressures and uses this to control the pressure and flow of gases and fluids delivered into the body cavity or hollow organ by an endoscope. A pressure transducer would be manufactured into the colonoscope tip, see FIG. 1. The connection would travel back through the colonoscope to the light source to a sensing module. The sensing module is able to be adjusted for different pressures. It would also have an audible alarm. The sensing module controls the activation module, see FIG. 2.

When the intra body cavity or hollow organ pressure is below the set pressure, the system delivers gases to the endoscope tip on demand. When the intra body cavity or hollow organ pressure is above the set pressure the delivery system is interrupted and an audible alert could be activated if the operator desired. When the intra organ pressure falls below the set level then flow would be re-established and the alert silenced. This protects the body from barotrauma.

Since there is more than one design of how gases and fluids are delivered to the body cavity or hollow organ by endoscopes, there has to be more than one method of turning off the flow by this feedback control system. The activation module could interrupt air flow by several mechanisms. These include, but are not limited to: turn off the electrical pump; vent the air pressure in the water bottle to atmosphere; close an electromechanical valve on the outflow of an external tank; close an electromagnetic valve to prevent delivery of gas or fluid to the body or override an electromagnetic demand valve used to allow flow in the first place.

1. Pressure transducers placed on the tip of an endoscope to sense intra cavity pressure and stop the flow of gas or fluid into the cavity when the pressure exceeds a pre-selected value will protect some patients from pressure-flow related injury.

2. An audible alarm when the pressure exceeds a pre-selected value will alert the operator that an unexpected potentially dangerous situation has occurred and he can take steps to correct it.

3. The endoscopic conduits used to deliver gas and fluid to the body cavity present resistance to their flow. Sufficient pressure is needed to overcome the resistance and provide adequate flow rates into the body cavity. Generally the faster the flow rate the more desirable the result, but the greater the chance for injury. This invention will allow powerful pressure sources to be used to accomplish desirable and purposeful tasks, yet insure the patient's body cavity is not exposed to dangerous pressure.
